(19) United States
(12) Patent Application Publication
(10) Pub. No.: US 2013/0158134 A1
(43) Pub. Date: Jun. 20, 2013

(54) Use of a safe, green and biodegradable absorptive material in the construction of diapers, feminine hygiene and adult incontinence products and devices

(71) Applicant: Samuel Kofele Martin, Burtonsville, MD (US)

(72) Inventor: Samuel Kofele Martin, Burtonsville, MD (US)

(21) Appl. No.: 13/585,860

(22) Filed: Aug. 15, 2012

Related U.S. Application Data
(60) Provisional application No. 61/575,708, filed on Aug. 25, 2011.

(51) Int. Cl. A61L 15/40; A61K 47/46

(52) U.S. Cl. CPC A61L 15/40; A61K 47/46
USPC 514/783; 604/367; 502/401

(57) ABSTRACT

A method of making absorbent core systems for the construction of absorbent articles such as baby care, feminine hygiene or adult incontinence products. The method essentially replaces the traditional synthetic acrylic acid polymer-based hydrophilic material mixed with wood pulp fiber in the absorbent core with a safer, green, biodegradable plant-based material.

### CROSS-REFERENCE TO RELATED APPLICATIONS

This patent application cross references U.S. Provisional Patent No. 61/575,708 of the same title, filed on 25 Aug. 2011 by Esingila Health and Wellness Consultants, Inc., 2916 Cabin Creek Drive, Burtonsville, Md. 20866.

None of the research for this work was done under federal government sponsorship.

### BACKGROUND OF THE INVENTION

The body discharges end products of its catabolism, digestion and other processes to the exterior. These substances frequently exit the body as liquids and semi-solids such as urine, feces, menses, sweat or gas. Despite the fact that the elimination of these products is essential to the house-keeping functions of the body, it simultaneously poses major hygiene challenges for appropriate socialization and acceptable decorum. Various methods have been developed to meet these social challenges. Methods have varied from shunning of the individual, frequent cleansing of the relevant discharge areas, application of antiperspirants and fragrances, to the more recent use of superabsorbent polymer- (SAP) based absorbent products like disposable tampons, feminine care pads, other catamenial devices, diapers and adult incontinence products. The earliest disposable products employed wood pulp fluff, cellulose wadding, fluff cellulose, or cotton fibers as the absorbent material. However, these materials did not absorb much moisture for their weight, and the resultant products were extremely bulky.

Even though the ancient Egyptians used softened papyrus to manage menstrual flow and other crude absorbent devices were already in use in Western Europe, credit for the modern tampon with applicator for insertion is given to Earle Hass who patented his device in the USA in November 1936 (U.S. Pat. No. 1,926,900). Commercial production and use of SAPs in the production of feminine hygiene products began in Japan in 1978 and by 1980 SAPs had been introduced and revolutionized diaper manufacture in Europe. Innovations in feminine hygiene and baby care products blossomed over time and attracted significant worldwide commercial interest particularly in the US. In keeping with demand, global SAP production capacity grew from only a few million metric tons in 1985 to greater than 700 million tons in 1995 with the United States accounting for 30% of this SAP demand.

The design of state-of-the-art feminine hygiene and related baby care and adult incontinence products has at its core an absorbent system with hydrophilic properties capable of trapping several times its weight in water. Despite the fact that plant products such as wood pulp fluff, cellulose wadding, fluff cellulose, or cotton fibers were the original absorbent materials, the industry quickly moved to synthetic acrylic acid derivatives, such as sodium, potassium or alkyl-acrylate, because of the much higher absorption ratios realized with these SAPs. However, the gains in absorbance with more efficient polymers mandated the use of fiber to overcome gel blocking. The added wood fibers act as thousands of tiny straws which suck up water faster and more efficiently and disperse it through the matrix to avoid gel blocking. Manufacturers have optimized the ratio of polymer and fibrous material that yield the most efficient absorbency and these products with SAP in their absorbent core enjoy the most success on the market because they are less bulky and trap water more efficiently without leakage of the collected body fluids.

Unfortunately, there are serious drawbacks to products and devices that use SAP-based/wood pulp fiber absorbent cores: 1. the acrylic acid based polymers are dependent on the chemical synthesis industry and add to the carbon footprint; 2. they are non-biodegradable and release potentially toxic products into the environment during manufacture, use and/or disposal; 3. an estimated 27.4 billion disposable diapers are used in the US each year adding about 3.4 million tons, about 1-3% of solid waste that go into the nation's landfills. As more countries around the world adopt these practices, the demand for SAPs and their environmental impact are projected to be huge. Furthermore, as life expectancy in the industrialized countries grow, the aging population will add increasingly more soiled SAP-core incontinence products to the landfills and underscore the urgent need for a solution to this looming and impending global crisis; 4. wood pulp fibers used to prevent gel blocking contain dioxin, a bi-product of the bleaching process of pulp with chlorine. Dioxin is a known human carcinogen. Arguably, these contaminants constitute a health hazard to both the users of these products and the environment when soiled items are disposed of either in community landfills, roadside dumps or sewage treatment facilities.

Practitioners of the prior art seems to be aware of these pitfalls and have introduced innovative products to counter some of these issues. In the year 2012 alone, several patents have been granted that relate to purported improvements in the design and manufacture of the absorbent core. Examples include (Baker et al U.S. Pat. No. 8,211,815 of 3 Jul. 2012—3-dimensional topography of top and bottom sheets; Seorens et al U.S. Pat. No. 8,207,395 of 26 Jun. 2012—absorbent core folded substrate; Ducker et al U.S. Pat. No. 8,202,609 of 19 Jun. 2012—absorbent material with wet strength containing wax; Malowaniec et al U.S. Pat. No. 8,202,390 of 18 Jun. 2012—disposable diaper open type; Venturino et al U.S. Pat. No. 8,198,506 of 12 Jun. 2012—stabilized composite material; Gartner et al U.S. Pat. No. 8,198,385 of 12 Jun. 2012—continuous batch production of SAP; Hakansson et al U.S. Pat. No. 8,183,430 of 22 May 2012—absorbent article with length width extension; Venturino et al U.S. Pat. No. 8,178,747 of 15 May 2012—absorptive core with elongate fluid holding formation. However, the emphasis on the innovations cited above has been on improving the intake, distribution and/or storage of the targeted fluids and not the elimination of SAPs. Some attention has also been given to cosmetic aspects such as comfort to wearer, (Van Himbergen et al U.S. Pat. No. 80,197,456 of 12 Jun. 2012—body adhering absorbent article; Busam et al U.S. Pat. No. 80,187,240 of 29 May 2012—thin and dry diaper) odor reduction (Do et al U.S. Pat. No. 8,168,563 of 1 May 2012—metal-modified silica particle for reducing odor; Wu et al U.S. Pat. No. 8,178,081 of 15 May 2012—method for reducing odor using polydentate compounds; Arehart et al U.S. Pat. No. 8,158,155 of 17 Apr. 2012—odor control cellulose-based granules) and packaging (Goates et al U.S. Pat. No. 8,162,913 of 24 Apr. 2012—folded absorbent article; Pateras et al U.S. Pat. No. 8,162,911 of 24 Apr. 2012—folded absorbent article). Even though much effort has been devoted to the redesign of the absorbent core system, the underlying issue of elimination of the SAP itself has been largely ignored. This invention recognizes the difficulties posed by SAPs in the manufacture, use and disposal of these products and outlines a simple and viable strategy to circumvent them.

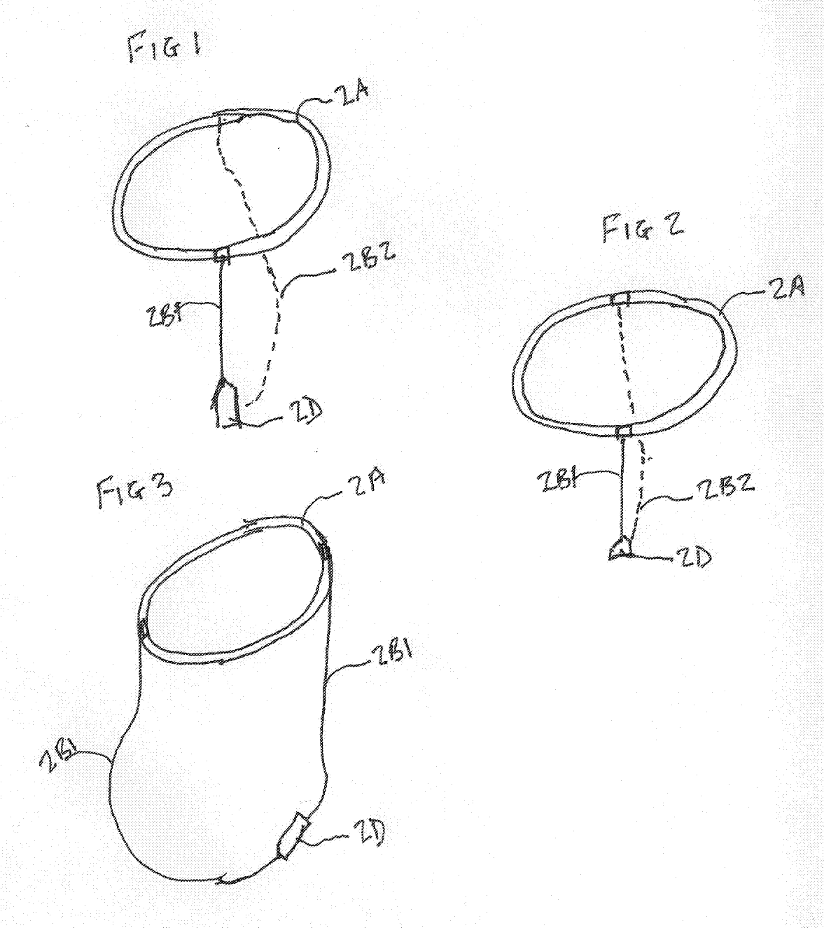

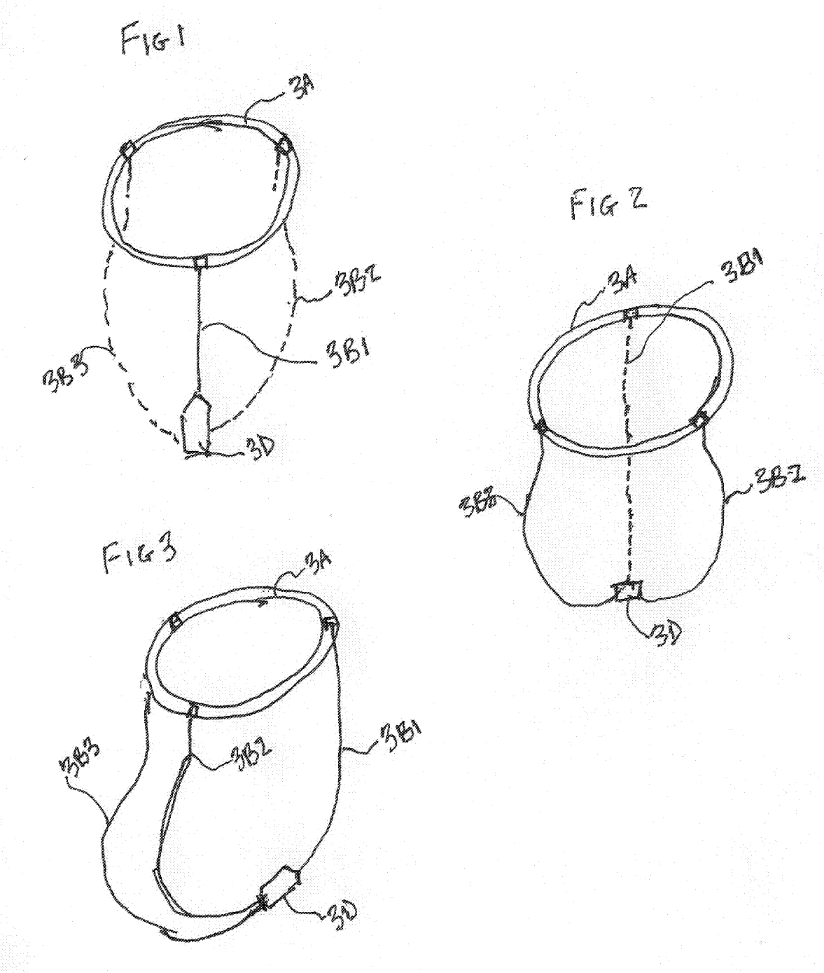

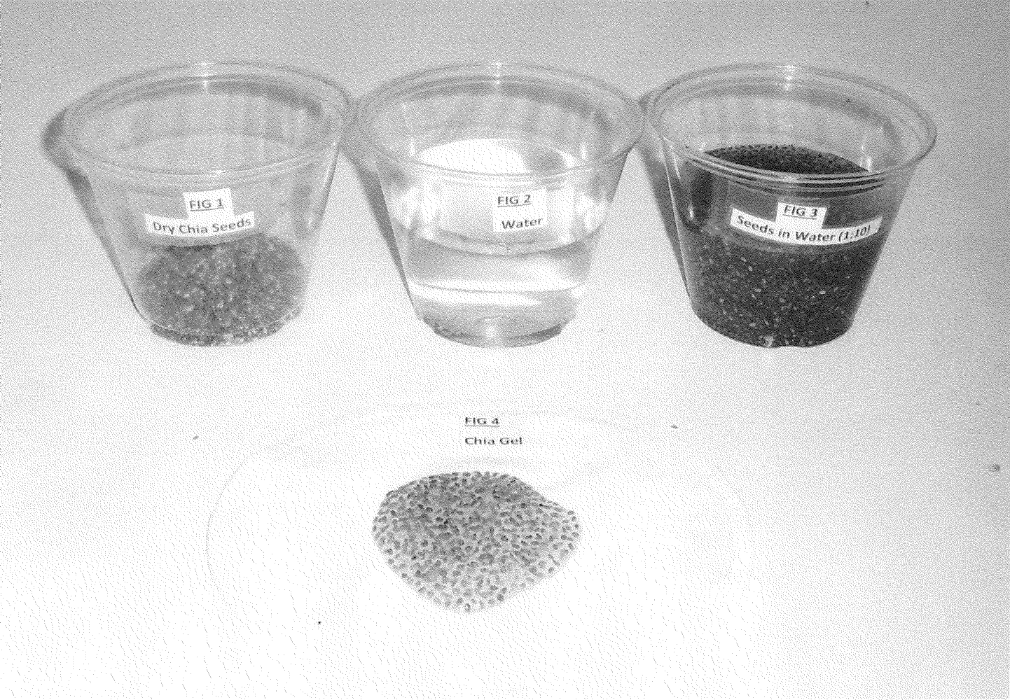

The mature seed of the plant Salvia hispanica is ingeniously designed to trap moisture. It comprises a highly hydrophilic core in the seed and displays an array of micro-fibers on its outer surface. Upon contact with moisture, the microfibers on each seed deploy and trap 10 to 15 times its weight in water, forming a stable gelatinous ring around the seed (Photo 1, FIG. 1, 2, 3). The trapped water surrounding the seeds coalesces to form a non-wetting gel (Photo1, FIG. 4). The microfibers serve a function not unlike that of pulp fibers manufactured into the absorbent material of the prior art to discourage gel blocking. Incredibly, the chia seed seems to have been configured by nature to provide the ideal absorbent material. Despite the fact that the seeds are biodegradable, taste-free, odor-free and so safe that it can be consumed by humans, its central role in the construction of absorbent articles particularly for human use had not been discovered. After absorbing water, wet chia seeds will quickly germinate if left in the presence of sunlight and can serve as seedlings for renewable chia cultivation, opening up the potential for recycling soiled chia seed absorbent core articles.

Norrby U.S. Pat. No. 8,206,365 of 26 Jun. 2012 instructs on the manufacture of a plant-type absorbent core. However, his invention combines cellulosic fluff pulp with SAP in the absorbent core and he renders no instructions for eliminating the SAP within the core of these products. Indeed, plant materials were the original absorptive material that was later replaced by a SAP core after the superior absorbent qualities of SAP were uncovered. Likewise, in the invention of Hird et al U.S. Pat. No. 8,198,505 of 12 Jun. 2012—the manufacture of non-bio-persistent inorganic vitreous microfibers; is described to improve the distribution and storage of water. Unfortunately, they also do not instruct on the crucial issue of eliminating the dependence on SAP at the absorbent core. Yet another attempt to redesign the absorbent core of absorbent articles is the development of absorbent gelling material. An example of this effort is illustrated in the work of Gagliadi Ivano et al Patent #20040058159 of Mar. 24, 2004. However, their patent does not claim to eliminate the SAP but rather teaches a method to suspend SAPs in particulate form in a liquid thermoplastic base material. The SAP particles are substantially angle-lacking and small to render the absorbent article optically transparent. This purportedly allows the wearer to wear see through clothing without focusing undue public attention to the absorbent article underneath their clothing. In spite of the present surge in interest and innovation within the industry, the intractable and mounting problems discussed above will persist until the present over-reliance and dependence of the prior art on SAPs is broken. This invention provides a viable alternative to the SAP-based absorbent core in proposing the use of chia seeds. They are biodegradable, safe, effective, plant-derived material that can functionally replace SAPs in the construction of highly absorbent baby care, feminine hygiene, adult incontinence and household products and devices.

### BRIEF SUMMARY

The substitution of SAP in the prior art with chia seeds will in practice engender the following advantages:

Biodegradability

SAP-based products are disposable but not completely biodegradable and their increased use globally is filling up municipal landfills. Disposable diapers are believed to constitute 1-3% of US and 15% of European bulkwaste, respectively, amounting to the annual production of millions of tons of garbage. A switch from the present products to biodegradable chia seed products will dramatically decrease the mounting tonnage of solid waste collected from households around the world.

Safety

As disposables captured a large share of the diaper market, questions have been raised about direct toxicity of the absorbent materials to consumers and to the general public after the soiled products are disposed into the environment. Polyacrylamide can cause skin allergy and lesions if they come into direct contact with the skin or mucous membranes. Moreover, the process of bleaching pulp with chlorine inevitably leads to the production of dioxin which is a known human carcinogen. Industry supporters argue that the bleached products are extensively washed to remove potentially hazardous substances. Even as these allegations remain unproven, they should cause concern because exposure to these products begin at infancy, are repetitive, intimate and unavoidable. A switch from SAP to chia seeds will lead to a change from a toxic chemical to a safe, edible and renewable plant material.

New Markets

There is a demand for products with enhanced absorbent capacity that remains unmet because of the relative toxicity associated with SAPs. In contrast, chia seeds are non-toxic, odor-free, taste-free and forms a stable gel when fully hydrated. Hence, the elimination of SAPs and introduction of chia seeds in the absorbent core should overcome some of the toxicity barriers and allow deeper penetration of these products into new markets within the food, health care, cosmetic and textile industries.

Reduced Dependence on the Chemical Industry

SAP-based products are dependent of the chemical synthesis industry for both of its major raw materials—acrylic acid derived polymers and chlorine bleached wood pulp fibers. It is estimated that about 27% of the production cost of diapers are attributable to the SAP. A switch to chia seed-based products will transform the industry from a chemical into a green industry that depends primarily on agriculture for its raw materials. The soiled chia seeds are completely biodegradable and may even be readily recycled. Therefore, swapping chia seeds for SAP in the absorbent core will drive production costs down and reduce the carbon footprint.

Green Agricultural Raw Materials

Chia seeds are presently cultivated for their nutritional value in the US, Central and South America and Australia to supply a limited novelty super-food niche market. Therefore, a new role for chia seeds in the baby care, feminine hygiene and incontinence products market, as intended by this invention, will drive up demand and lead to a greening of presently under utilized semi-arid lands around the world that are best suited for their cultivation. Furthermore, it will also create an incentive for innovation to improve large scale organic farming, genetic engineering and breeding for stocks with higher hydrophilic and other properties. Such activity will optimize yields, reduce cost and reduce dependency on related carbon-based manufacturing processes. Indeed, the attendant mainstreaming of the human use of chia for their absorbent properties, initiated by this invention, will simultaneously popularize its use as a food source around the globe.

### BRIEF DESCRIPTION OF THE INVENTION

The use of the term, chia seeds, in this invention covers mature dry seeds of the mint (sage) plant family Lamiaceae, species Salvia hispanica, Salvia columbariae and other natural or man-made genetically modified varieties.

The term superabsorbent polymers (SAP) is used in this invention to cover all acylic acid derived polymers and combinations thereof, synthesized for their hydrophilic properties and utilized in the construction of absorbent articles to include baby care, feminine hygiene and adult incontinence products and devices.

Photograph 1 demonstrates the water trapping properties of chia seeds. Ten volumes of water were added to one volume of seeds and mixed for 20 minutes to form a gel. A portion of the gel was placed on cloth and photographed after one hour. Similar results were obtained using human urine in place of water.

FIG. 1 One volume of dry chia seeds in a plastic cup.

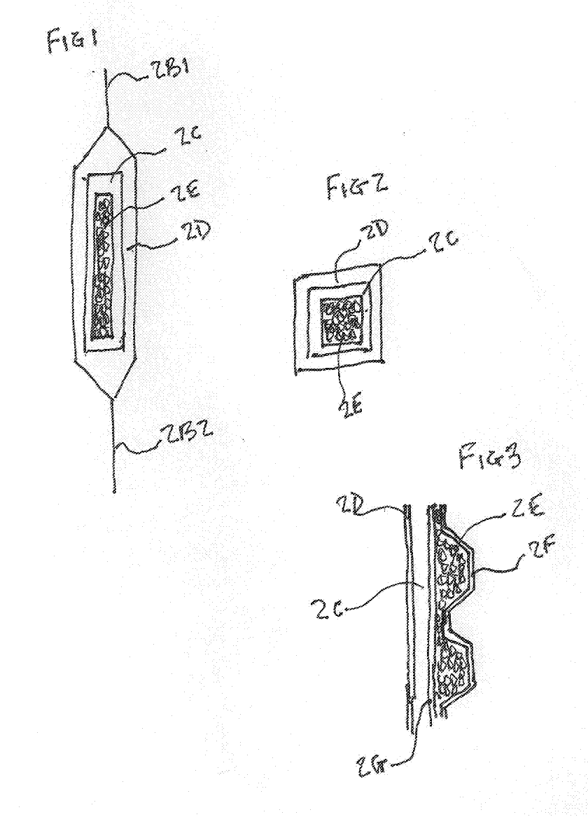

FIG. 2 Ten volumes of water in a similar plastic cup.

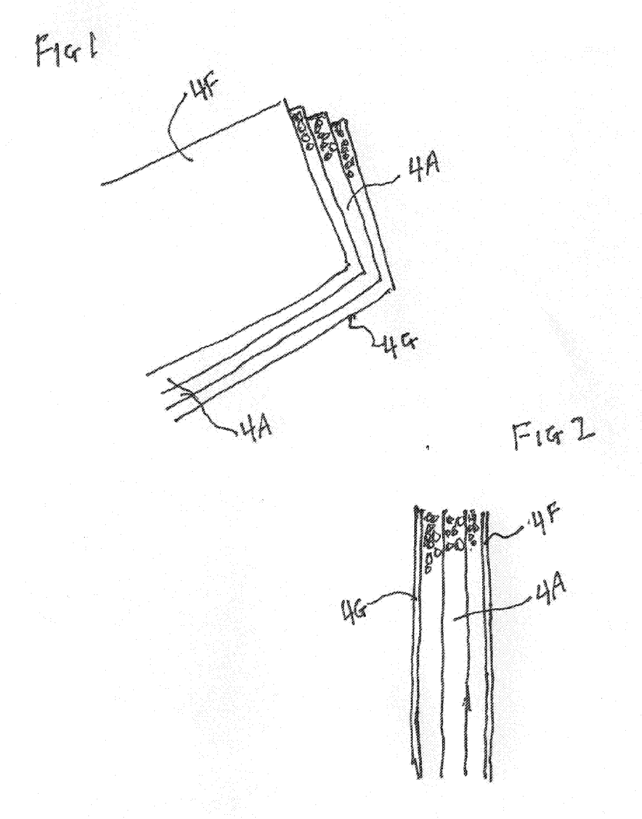

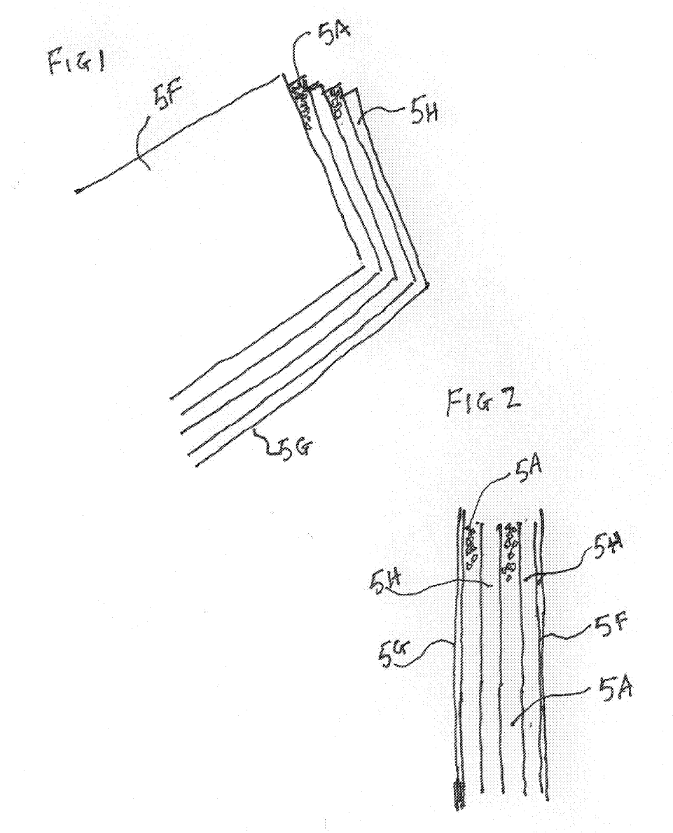

FIG. 3 Ten volumes of water mixed with one volume of dry chia seeds in a similar plastic cup.

FIG. 4 Gel placed on white cloth. Trapped water in gel does not wet cloth.

SKETCH 1(A, B, C and D): Sketch of first preferred embodiment with cradle (2D) suspended on belt (2A) via an anterior sling (2B1) and posterior sling (2B2) showing—anterior (FIG. 1), posterior (FIG. 2) and lateral (FIG. 3) views

SKETCH 2(A, B1 B2 B3, C and D): Sketch of second preferred embodiment with cradle (3D) suspended on belt (3A) via a combination of slings (3B1, 3B2, 3B3) showing—anterior (FIG. 1), posterior (FIG. 2) and lateral (FIG. 3) views

SKETCH 3: Sketch of a pad mounted on cradle with views showing frontal (FIG. 1) and cross-sectional cut (FIGS. 2 & 3) through the assembly.

SKETCH 4: Sketch of third preferred embodiment with chia seeds impregnated directly into material with views showing frontal (FIG. 1) and cross sectional cut (FIG. 2) of the sheet assembly

SKETCH 5: Sketch of third preferred embodiment with chia seeds impregnated directly into material and wicking material interposed between sheets of chia seeds impregnated material with views showing frontal (FIG. 1) and cross sectional cut (FIG. 2) of sheet assembly

### DETAILED DESCRIPTION OF THE INVENTION

In general, absorbent articles of the prior art comprise a SAP-based absorbent core into which bleached wood pulp fibers have been introduced to prevent gel blocking. This core is embedded between a layer(s) of water permeable sheet(s) on the wearer-side and a water impermeable sheet on the other side of the article. The layers are fused together by heat or ultrasound to form the fluid absorbent article. Variations abound around this basic design engineered to improve absorption, distribution and trapping of the targeted body fluid while discouraging bulking, roping and/or leakage. Accordingly, the general practice of this invention instructs a simple swap of the SAP or other absorbent core of the prior art with the chia seed absorbent core of this invention. Modifications in the art needed to replace the SAP absorbent core are covered under specific preferred embodiments discussed below.

A first preferred embodiment of this invention is illustrated in Sketch 1 (FIGS. 1, 2 and 3). The embodiment has an adjustable belt (2A) with a device to size fit the belt around the waist of the user. The belt possesses two attachment elements that deploy in the midline to the front and back of the user when the belt is worn. The belt can be made of any material to include, fabric, elastic, rubber, leather etc to accommodate the preferences of the user. Two slings (2B1, 2B2) with a fastening element at one end and the other end permanently attached to a cradle element (2D) suspend the cradle on the belt. The pad or absorbent article (2C) is designed to be detachably affixed within the cradle in order to gain optimal fit and contact to the area(s) of origin of the targeted body fluid. The sling (2B2) runs backwards from its attachment at the back end of the cradle (2D) between the buttocks and upwards to attach to the posterior attachment element on the belt. The anterior sling (2B1) runs from its attachment at the front end of the cradle upwards to the anterior attachment element on the front of the belt. The absorbent article or pad is detachably mounted within the cradle. The belt, cradle and slings are designed for multiple-use and are not disposed of with the soiled pad but cleansed as appropriate for re-use with a fresh pad. The pad comprises an absorbent core of chia seeds (2E) embedded within a water permeable front sheet (2F) and water impervious back sheet (2G) all made of biodegradable materials. Additionally, the slings are made of expandable material or provided with a mechanism to size fit the device to the wearer's specifications.

In another preferred embodiment of the invention, Sketch 2 (FIGS. 1, 2 and 3) the sling (3B1) attaches the front end of the cradle (3D) to the belt. Two slings (3B2 and 3B3) attach the back end of the cradle to the belt by passing over each buttock rather than between the buttocks as instructed in the first embodiment. The cradle is modified to allow for detachable fixture and support of the absorbent article (3C). The slings can be permanently attached to the belt and cradle and worn by stepping through the loops formed by the attachment points of the slings to the belt and cradle. Allowance is made on the belt and slings to size fit for body habitus, comfort and esthetic preferences of the wearer. For example, the slings can be modified to cover the buttocks and/or lower abdomen, like panties or underwear. The slings can also be modified to pass over the anterior iliac crests and attach to the posterior attachment devices on the belt. In operation, the slings suspend the cradle that carries the pad or collection article and holds it in close proximity to the discharge opening of the targeted body fluid. Adhesive material can be used to detachably affix the pad to the cradle (3D) to improve support and stability, allow maneuverability of the wearer and limit leakage. The cradle and pad can be modified and contoured (FIG. 3) to meet the esthetic and comfort preferences of the user. As in the first embodiment, the pad has an absorbent core of chia seeds (3E) embedded within a top (3F) and bottom (3G) sheet. The belt, slings and cradle can be made of material that allow for appropriate cleansing and re-use whereas the absorbent article or pad is composed of biodegradable materials to be discarded after single use. The chia seed absorbent core can be detachably affixed to the bottom sheet when a suitable biodegradable impervious material cannot be sourced. This ensures that the soiled article is completely biodegradable upon disposal.

In a third embodiment of this invention, the chia seeds or derivatives therefrom are impregnated into tissue or fabric with the seeds adherent to the constituent fibers or threads of the material (4A). The impregnated sheets are cut and layered between a top permeable (4F) and water impervious bottom sheet (4G). In a fourth embodiment, a wicking element (5H) is interposed between the seed impregnated sheets (5A) to pull moisture away from the top sheets for dryness towards the bottom sheet for distribution and storage. The (5A/5H/5A/5H . . . ) assembly is then embedded between a top and bottom pervious sheet or a top pervious and bottom impervious sheet dependent on the intended purpose of the absorbent article. Such layered materials will have a multiplier effect on absorbency and are particularly suited for the construction of disposable diapers, feminine hygiene products and under-garments that can be used for the collection and disposal of urine, menses and sweat, respectively. Other uses for articles informed by the third and fourth embodiments include but are not limited to surgical sponges, sweat pads or bands, wipes for baby care and other domestic needs, wipes for picking up spills at home and at the work place.

An additional embodiment of this invention takes advantage of the stable gel formed upon hydration of chia seeds. Chia seeds are packaged with other compounds or substances such as a fragrance, antigens, vaccines or drug for encapsulation upon hydration. Water is added to the mixture to form a gel prior to use. The gel prevents the rapid release of the trapped additive when swallowed. This embodiment is of particular interest in drug, vaccines and antigen delivery systems where the deliverable has a disagreeable taste or is sensitive to conditions in the stomach and upper gastrointestinal tract. The gel may also be used to target specific cells within the lining of the gut for binding and subsequent uptake of the additive.

The average chia seed measures about 2.1×1.3 mm about the size of a poppy seed or pin head. They are non-allergenic, non-toxic, odor-free, taste-free and form a stable gel after trapping water. The relatively large size and non-toxic nature of the seeds compared to the powdery texture and toxicity of SAPs widens the choice of materials for the top and back sheets. Hence, advantage should be taken of these properties to ensure that materials are chosen that optimize the flow of the targeted body fluids into the pad, maximize the comfort and preferences of the users and more importantly, optimize the biodegradability of the soiled pad or absorbent articles. Furthermore, the relatively large size and firmness of chia seeds compared to SAPs of the prior art can be exploited to improve contact between the absorptive pad and the vulva to minimize leakage and discourage roping in the collection of menses, (Sketch 3, FIG. 3). The chia seeds will swell as they retain liquid thereby further improving fit and preventing leakage. In the prior art, the liquid pervious top sheets and liquid impervious back sheets are fused to the absorbent core using heat or ultrasound. In these improved embodiments, allowance is made to separate the chia seeds absorbent core from the back sheets if needed to render the detached soiled pad completely biodegradable at disposal.

1. The use of seeds or derivatives therefrom of Salvia hispanica and related species of the mint plant family Lamiaceae (chia seeds) partially or exclusively as the absorbent core in the construction of baby care, feminine hygiene and adult incontinence products and devices for the hygienic collection and disposal of body and other fluids or spillage.

2. The addition of chia seeds or derivatives therefrom to solids, liquids, woven and non-woven fabric, tissue and the like to enhance their absorbent capacity for water and fluids to include body fluids such as urine, feces, menses, discharges, sweat, saliva, tears, pus, exudates and other fluids or spillage.

3. The use of chia seeds or derivatives therefrom in delivery systems to improve compliance or the bioavailability of drugs, vaccines, antigens etc or to target cells in vitro or in vivo
